(19) United States
(12) Patent Application Publication
(10) Pub. No.: US 2014/0309631 A1
(43) Pub. Date: Oct. 16, 2014

(54) FLATTENED MESH ABLATION DEVICE

(71) Applicant: Cook Medical Technologies LLC, Bloomington, IN (US)

(72) Inventors: Tyler Evans McLawhorn, Winston-Salem, NC (US); Vihar C. Surti, Winston-Salem, NC (US)

(73) Assignee: Cook Medical Technologies LLC, Bloomington, IN (US)

(21) Appl. No.: 14/212,268

(22) Filed: Mar. 14, 2014

Related U.S. Application Data
(60) Provisional application No. 61/798,164, filed on Mar. 15, 2013.

(51) Int. Cl. A61B 18/14; A61B 18/12

(52) U.S. Cl. CPC A61B 18/1492; A61B 18/1206; A61B 2018/1437; A61B 2018/1465
USPC 606/34

(57) ABSTRACT

A flattened mesh ablation device for ablating tissue in a body lumen is disclosed. The flattened mesh ablation device includes a flattened mesh with at least one conductor on an edge of the flattened mesh. When the flattened mesh is compressed axially it expands radially to contact the inner surface of the body lumen in a helical pattern. Energy is applied to the conductor ablating tissue proximate the conductor.

### RELATED APPLICATIONS

The present patent document claims the benefit of the filing date under 35 U.S.C. §119(e) of Provisional U.S. Patent Application Ser. No. 61/798,164, filed Mar. 15, 2013, which is hereby incorporated by reference.

### FIELD

This invention relates generally to medical devices for ablating tissue in a body lumen. More particularly, this invention relates to a system for ablating tissue in a wall of a blood vessel.

### BACKGROUND

Hypertension, commonly referred to as high blood pressure is typically treated using antihypertensive medication. However, there is a patient population that is unresponsive to this pharmacological approach and other approaches have been developed to treat hypertension.

Blood pressure has been shown to be partially controlled by the kidneys and renal sympathetic nerve hyperactivity has been linked to hypertension. Recently, intravenous catheter based technologies have been developed to disrupt the sympathetic nervous system surrounding the renal arteries. These intravenous catheter technologies use an energy source to ablate the tissue around the renal artery. Two energy sources being used to ablate the tissue and disrupt these nerves are radiofrequency (RF) and ultrasound.

The sympathetic nervous system fully encapsulates the renal artery so to be fully effective, a full 360 degree ablation is necessary. However, with the RF systems, a circular ablation at a single location can damage the lining of the renal artery such that the lumen strictures, or narrows, thus reducing blood flow to the kidneys. To avoid stricturing, the currently available RF systems ablate a helical section of tissue such that 360 degrees of tissue is treated over a much longer section of a vessel.

One current system uses a balloon platform where a flexible electrode forms a helix on the surface of the balloon. The user guides the balloon to the treatment site and inflates the balloon such that the electrode contacts the target tissue. With this system, the entire ablation can take place with a single application. However, since the system is balloon based, blood flow is blocked for the duration of the ablation procedure. Additionally, as it is balloon based, the size of the balloon will have to closely match the size of the target vessel to ensure adequate tissue/electrode contact without over extension of the vessel.

In another current system, an electrode is mounted on the distal end of a deflecting catheter. The user deflects the tip of the catheter with the electrode and ablates a section of the vessel. The tip is then moved axially and the catheter rotated to ablate another section of the vessel. This is repeated at 3-4 locations working from distal to proximal while continuing to rotate the catheter approximately ¼ turn at each new site. Energy is dispersed at each independent site for approximately 2 minutes to ablate the tissue, for a total treatment time of 8 minutes for the ablation.

The balloon system described previously is faster than the deflecting catheter system described since it only needs to disperse energy a single time to ablate a 360 degree section of the vessel. However, the deflecting catheter system is preferable since it does not stop the flow of blood through the body lumen. It would be beneficial to have a system that combines the speed of the balloon based system while still allowing blood to flow through the vessel like the deflecting catheter system.

### SUMMARY

Embodiments of the invention include a medical device comprising a first longitudinal member, a flattened mesh, a conductor, and a compression mechanism. The first longitudinal member has a distal end and a proximal end. The flattened mesh has a distal mesh end and a proximal mesh end and is twisted to form a helix. The proximal mesh end is secured to the distal end of the first longitudinal member. The conductor is disposed on an edge of the helix. The compression mechanism is adapted to move the distal mesh end between a first position in which the flattened mesh is unexpanded and a second position in which the distal mesh end and the proximal mesh end are near one another thereby expanding the flattened mesh into an expanded state.

In another embodiment a medical device comprises a catheter, a flattened mesh, a conductor, and a sleeve. The catheter has a distal end and a first outer diameter at the distal end. The flattened mesh has a distal mesh end and a proximal mesh end and is biased to a helical shape having a first helix outside diameter greater than the first outside diameter. The proximal mesh end is secured to the distal end of the catheter. The conductor is disposed on an edge of the helical shape. The sleeve is disposed about the distal end of the catheter and has an inside surface with an inside diameter greater than the first outside diameter and less than the helix outside diameter. The sleeve is slidable from a first position in which the inside surface constrains the flattened mesh to have a second helix outer diameter less than the second outer diameter and a second position in which the inside surface does not constrain the flattened mesh.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 illustrates a longitudinal cross section of an embodiment of a flattened mesh ablation device.

FIG. 2 illustrates a longitudinal cross section of the embodiment of the flattened mesh ablation device of FIG. 1 with the device in an expanded configuration.

FIG. 3 illustrates a cross-section of FIG. 2 taken at line 3-3.

FIG. 4 illustrates a longitudinal view of a flattened mesh.

FIG. 5 illustrates a longitudinal view of an embodiment of a flattened mesh showing the placement of a conductor.

FIG. 6 illustrates the flattened mesh of FIG. 5 in an expanded configuration.

FIG. 7 illustrates a longitudinal view of an embodiment of a flattened mesh showing the placement of a pair of conductors.

FIG. 8 illustrates the flattened mesh of FIG. 7 in an expanded configuration.

FIG. 9 illustrates a longitudinal view of an embodiment of a flexible mesh showing the placement of a pair of conductors.

FIG. 10 illustrates the flattened mesh of FIG. 9 in an expanded configuration.

FIG. 11 illustrates a proximal end of a flattened mesh ablation device.

FIG. 12 illustrates another embodiment of a flattened mesh ablation device.

FIG. 13 illustrates the embodiment of a flattened mesh ablation device of FIG. 12 with a sheath constraining the flattened mesh.

The drawings are not necessarily to scale.

### DETAILED DESCRIPTION

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Detailed Description does not contain all of the aspects and embodiments of the one or more present inventions, is not meant to be limiting or restrictive in any manner, and that the invention(s) as disclosed herein is/are and will be understood by those of ordinary skill in the art to encompass obvious improvements and modifications thereto.

Additional advantages of the present invention will become readily apparent from the following discussion, particularly when taken together with the accompanying drawings.

In the following discussion, the terms “proximal” and “distal” will be used to describe the opposing axial ends of the inventive ablation device, as well as the axial ends of various component features. The term “proximal” is used in its conventional sense to refer to the end of the ablation device (or component thereof) that is closest to the operator during use of the ablation device. The term “distal” is used in its conventional sense to refer to the end of the ablation device (or component thereof) that is initially inserted into the patient, or that is closest to the patient during use. For example, an ablation device may have a proximal end and a distal end, with the proximal end designating the end closest to the operator, such as a handle, and the distal end designating an opposite end of the ablation device. Similarly, the term “proximally” refers to a direction that is generally towards the operator along the path of the ablation device and the term “distally” refers to a direction that is generally away from the operator along the ablation device.

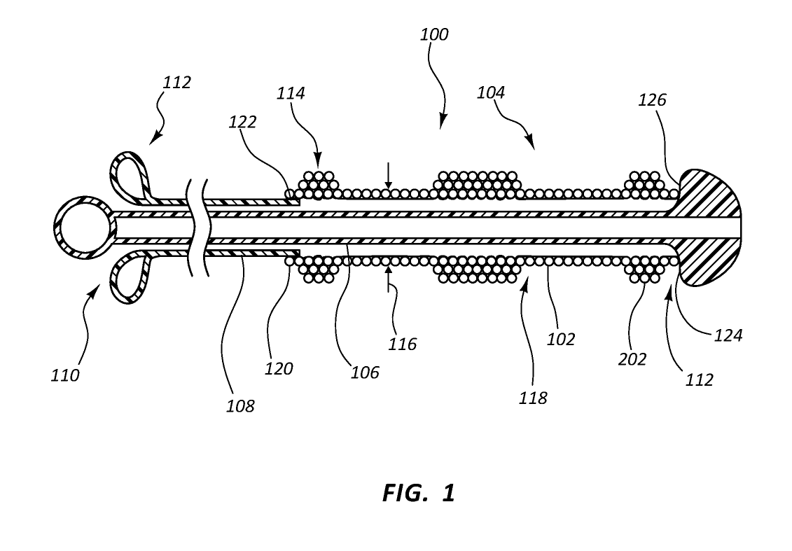

FIG. 1 illustrates an embodiment of a flattened mesh ablation device 100 in accordance with the present invention. The flattened mesh ablation device 100 includes a flattened woven mesh 102 at a distal portion 104. The flattened woven mesh 102 is twisted to form a helix. The helix shown in FIG. 1 has one turn, although different numbers of turns are possible. The flattened woven mesh 102 is operably connected to an inner shaft 106 and an outer shaft 108. The flattened woven mesh 102 may be secured at a proximal end 120 to a distal end 122 of the outer shaft 108 and at a distal end 124 to a distal end 126 of the inner shaft 106. The flattened woven mesh 102 may be twisted in a helical shape by radially offsetting the distal end 122 of the outer shaft 108 and the distal end 124 of the inner shaft 106. The outer shaft 108 and the inner shaft 106 may be keyed to prevent rotation, maintaining the helical shape of the flattened woven mesh 102. In other embodiments the inner shaft 106 is free to rotate relative to the outer shaft 108. In such embodiments it may be necessary for the operator to apply a torque to one of the shafts to twist the flattened woven mesh 102 into a helical shape.

In some embodiments, the inner shaft 106 is coaxially positioned within the outer shaft 108 as shown in FIG. 1. The flattened woven mesh 102 expands and collapses by longitudinal movement of the inner shaft 106 relative to the outer shaft 108 as explained in more detail below. A control handle 110 is provided at a proximal portion 112 of the flattened mesh ablation device 100. The control handle 110 is operable to control the movement of the inner shaft 106 and the outer shaft 108 relative to one another. The control handle 110 may be any type of handle that is operable to control the movement of the inner shaft 106 relative to the outer shaft 108 and need not have the structure illustrated in FIG. 1.

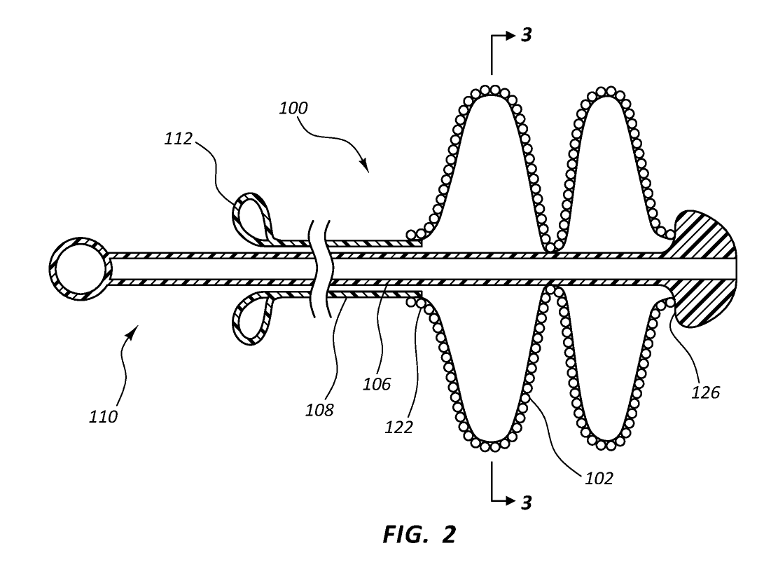

As shown in FIG. 1, a distal portion 112 of the flattened woven mesh 102 is operably connected to the inner shaft 106. A proximal portion 114 of the flattened woven mesh 102 is operably connected to the outer shaft 108. Relative movement between the inner shaft 106 and the outer shaft 108 causes the flattened woven mesh 102 to change between a collapsed configuration shown in FIG. 1, and an expanded configuration shown in FIG. 2. The flattened woven mesh 102 in the unexpanded configuration has a first outside diameter 116 and the flattened woven mesh 102 in the expanded configuration extends beyond the first outside diameter 116 at a middle segment 118. The unexpanded configuration may be used to deliver the flattened mesh ablation device 100 to a treatment site within a patient and for repositioning the flattened mesh ablation device 100 within a patient's lumen to provide treatment to additional sites if needed.

FIG. 2 illustrates the flattened mesh ablation device of FIG. 1 in an expanded configuration. The outer shaft 108 has been moved distally relative to the inner shaft 106 decreasing the distance between the distal end 122 of the outer shaft 108 and the distal end 126 of the inner shaft 106. The decrease in distance causes the flattened woven mesh 102 to expand radially as shown in FIG. 2.

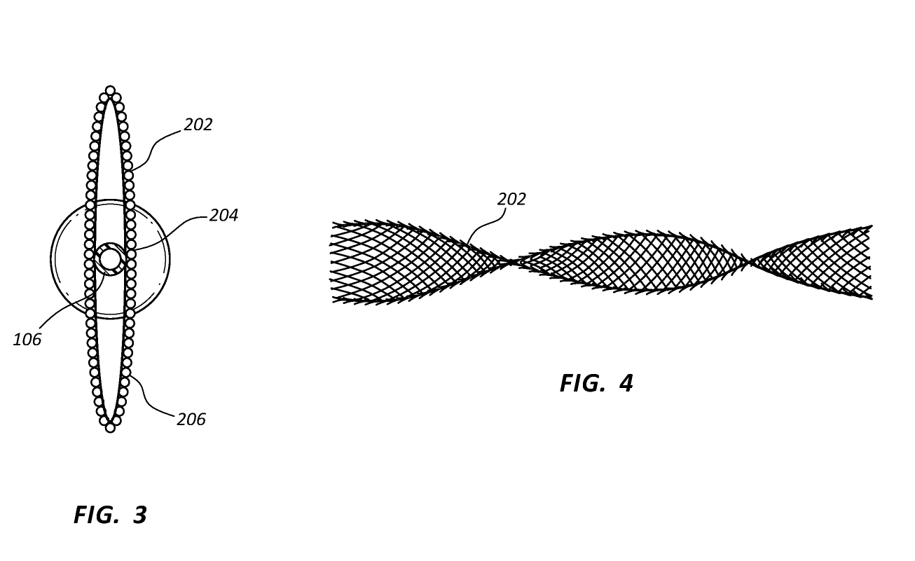

A cross-sectional view of the flattened mesh 102 of FIG. 2 taken at line 3-3 102 is shown in FIG. 3 and a side view of the flattened woven mesh 102 is shown in FIG. 4. The flattened woven mesh 102 is comprised of a plurality of filaments 202 that are woven together to form a cylindrical sleeve that is then flattened to have a flattened inner surface 204 and a flattened outer surface 206. In some embodiments, the filaments 202 may be formed from a nonconductive material such as a polyolefin, a fluoropolymer, a polyester, for example, polypropylene, polytetrafluoroethylene, polyvinylidene fluoride, polyethylene terephthalate (PET), and combinations thereof. Other materials known to one skilled in the art may also be used to form the filaments 202, provided that they enable the flattened woven mesh 102 to be changeable from the expanded state and the unexpanded state in response to the inner shaft 106 moving relative to the outer shaft 108.

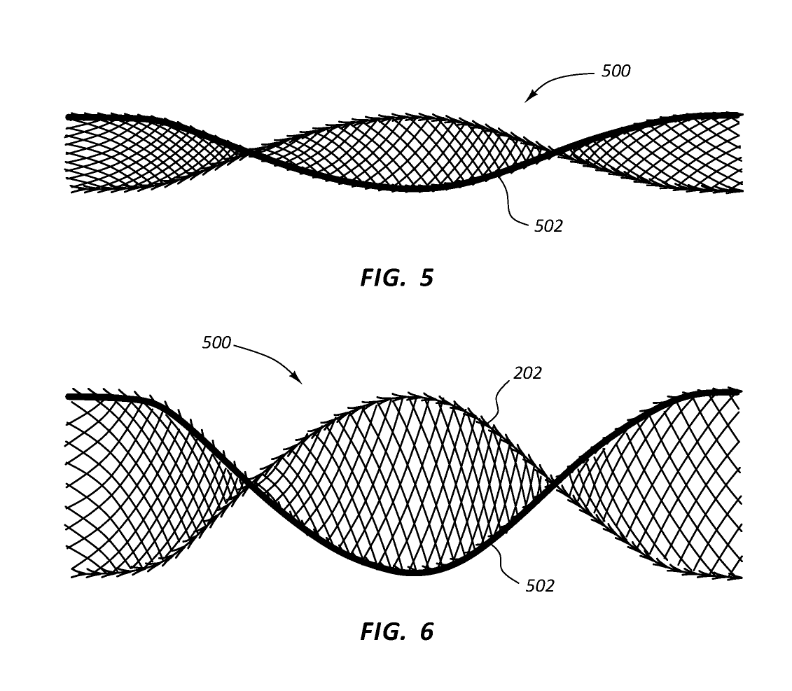

FIG. 5 through FIG. 9 illustrates various embodiments of a flattened woven mesh 102 suitable for use in the flattened mesh ablation device shown in FIG. 1. The inner shaft 106 and outer shaft 108 are not illustrated for clarity. Each embodiment of the flattened woven mesh 102 will be illustrated in an unexpanded state and an expanded state. It will be generally understood that the flattened woven mesh 102 may enter the expanded state when the proximal end 120 and the distal end 124 of the flattened woven mesh 102 are brought closer together.

FIG. 5 illustrates an exemplary flattened woven mesh 500 illustrating the placement of a conductor on an edge 504 of the flattened woven mesh 500. The conductor may comprise a conductive coating 502 when the mesh is comprised of nonconductive filaments. In other embodiments, the conductor may comprise one or more conductive filaments braided into the flattened woven mesh 500 in the pattern of the conductive coating. FIG. 6 illustrates the same flattened woven mesh 500 as FIG. 5, but with the flattened woven mesh 500 being expanded. In this embodiment the conductive coating 502 is applied to the edge 504 of the flattened woven mesh 500 to form a helical conductor.

In some embodiments, the conductive coating 502 may span a gap between adjacent filaments. A flexible base material may be attached the edge 504 of the mesh as a base layer for the conductive coating 502. The flexible base material may span the area between filaments which may increase the amount of conductive coating 502 that can be applied. One example of a suitable flexible base material between the conductive coating 502 and the filaments is silicone.

The conductive coating 502 may be a conductive ink applied to the surface of the mesh. One example a conductive ink is silver ink, although other metallic inks are possible. The conductive coating 502 may comprise a conductive painting, conductive glue, or other conductive materials that form a flexible coating on the non-conductive filaments 504 without spanning the gap between adjacent filaments.

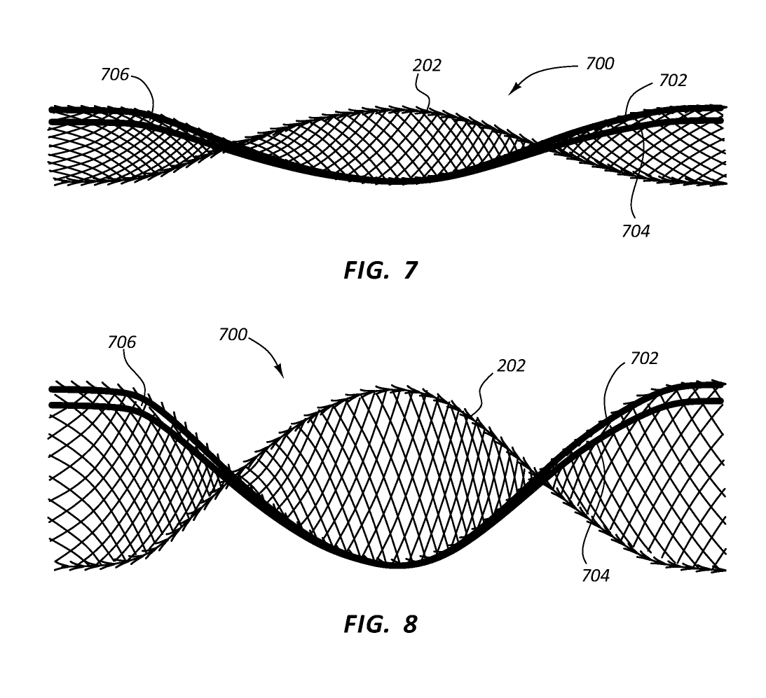

FIG. 7 illustrates an exemplary flattened woven mesh 700 illustrating a bipolar arrangement of conductive coatings 702, 704. FIG. 8 illustrates the flattened woven mesh 700 of FIG. 7 with the flattened woven mesh 700 being expanded. A first conductive coating 702 coats a first portion of an edge 706 of the flattened woven mesh and a second conductive coating coats a second portion of the edge 706. An ablation zone 708 is formed between the first conductive coating 702 and the second conductive coating 704. Because the filaments of the flattened woven mesh 700 are non-conducting, there is a high electrical resistance between the first conductive coating 702 and the second conductive coating 704. This bipolar arrangement allows for a precise ablation zone 708 between the conductive coatings 702, 704.

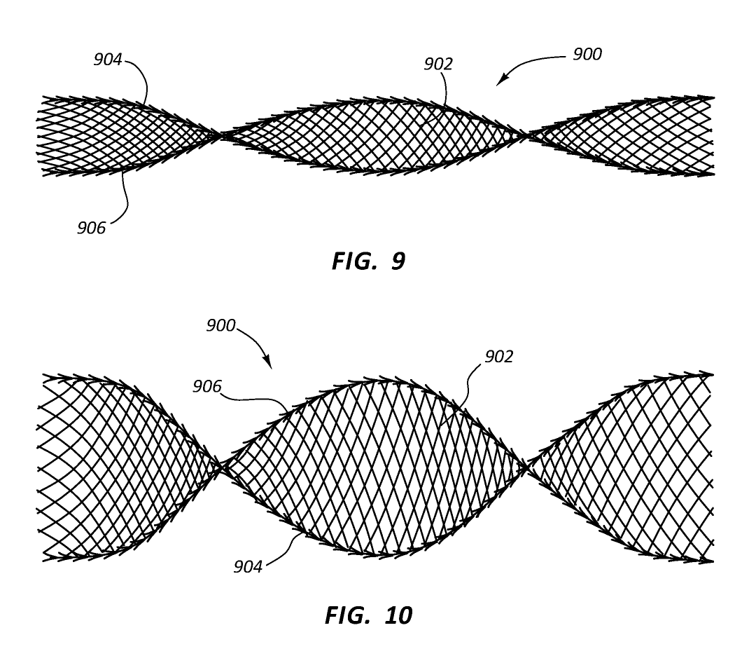

FIG. 9 illustrates another embodiment of an exemplary flattened mesh 900 illustrating another pattern of conductive coatings to form an ablation device. FIG. 10 illustrates the flattened mesh 900 of FIG. 9 with the flattened mesh 900 expanded. In the embodiment of FIG. 9 a conductive coating 902 coats the entire surface of the flattened mesh 900. Because the flattened mesh is twisted into a helix, only a first edge 904 and a second edge 906 contact the inner surface of a lumen. Ablation will only occur where the edges 904, 906 contact the inner surface and 2 helical ablation patterns result. In this particular embodiment, the flattened mesh may be woven using conductive filaments with no conductive coating required.

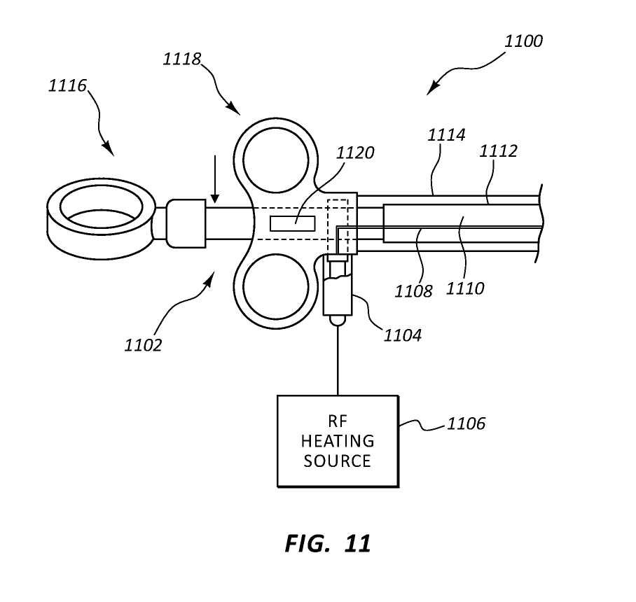

FIG. 11 illustrates the proximal end of a flattened mesh ablation device 1100. In each of the previously described embodiments, the conductive coating is operably connected to an energy source. As shown in FIG. 11, a handle 1102 may include a connector 1104 for operably connecting the conductive coating to an energy source 1106. As shown, the energy source 1106 may be a radio frequency source. However, other types of energy sources may also be used to provide energy to the conductive coating. By way of non-limiting example, additional possible energy sources may include microwave and electric current. The conductive coating is connected to the power source by an electrical conductor, such as one or more wires 1108 that extend from the conductive coating to the connector 1104 that connects to the energy source 1106. The one or more wires 1108 may extend through a lumen 1110 of the inner 1112 shaft or may extend through a lumen of the outer shaft 1114 or external to the outer shaft 1114 and may optionally include a sleeve surrounding the outer shaft 1114 and one or more wires 1108.

As discussed above, the handle 1102 is operable to move the inner shaft 1112 relative to the outer shaft 1114 so that the flattened woven mesh 1102 moves between the expanded configuration and the collapsed configuration (see FIGS. 1 and 2). By way of non-limiting example, the handle 1102 includes a first portion 1116 and a second portion 1118 that move relative to each other. As shown in FIG. 11, the first portion 1116 is operably connected to the inner shaft 1112. The second portion 1118 is operably connected to the outer shaft 1114. The first portion 1116 may be moved proximally and/or the second portion 1118 may be moved distally to move the inner shaft 1112 proximally and/or the outer shaft 1114 distally to move the flattened woven mesh 102 to the expanded configuration as shown in FIG. 2. As shown in FIG. 1, the first portion 1116 may be moved distally and/or the second portion 1118 moved proximally to move the inner shaft 1112 distally and/or the outer shaft 1114 proximally to move the flattened woven mesh 102 to the collapsed configuration.

The handle 1102 may include a lock 1120 shown in to releasably lock the first portion 1116 in position relative to the second portion 1118 and thus lock the flattened woven mesh 102 in position. The lock 1120 may releasably lock the first and second portions 1116, 1118 of the handle 1102 together at any proximal/distal positioning of the inner and outer shafts 1112, 1114 so that the flattened woven mesh 102 may be locked at any size that is suitable for the treatment site. For example, if the treatment site is in a narrow lumen, the first portion 1116 of the handle 1102 may be moved slightly in the proximal direction to give the flattened woven mesh 102 a smaller diameter than if the first portion 1116 were moved fully distally to give the flattened woven mesh 102 the largest diameter.

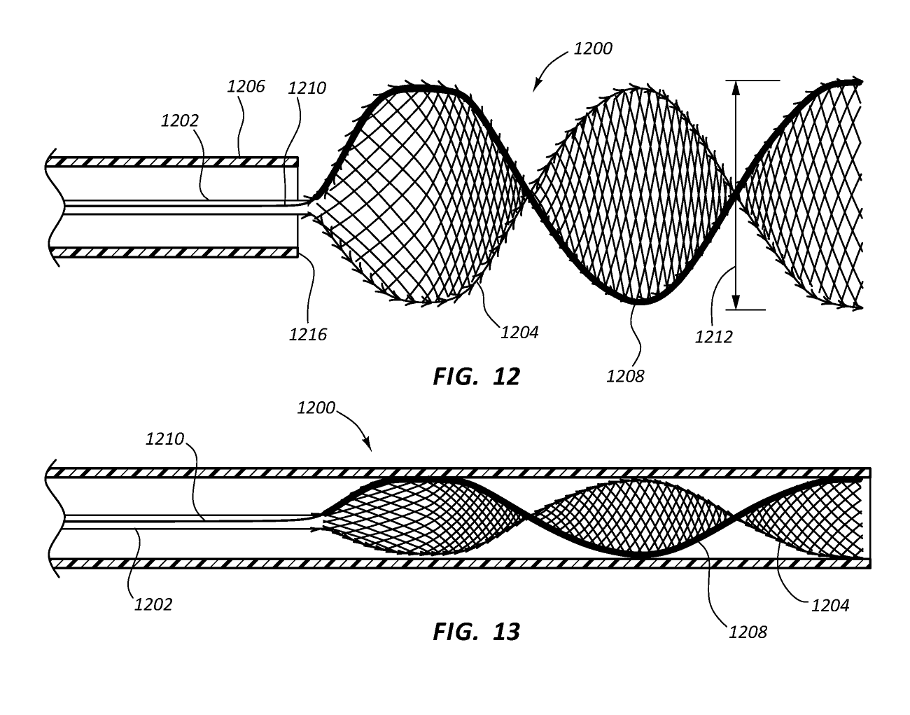

FIG. 12 illustrates another embodiment of a flattened mesh ablation device 1200. The flattened mesh ablation device 1200 is comprised of a catheter 1202, a flattened mesh 1204, and a sheath 1206. The sheath 1206 is mounted about the catheter 1202 such that it may be moved between a first location (shown in FIG. 13) in which the sheath 1206 provides a radial constraint to the flattened mesh 1204 and a second position (shown in FIG. 12) in which the sheath 1206 does not provide a radial constraint to the flattened mesh 1204. A conductive coating 1208 is disposed on an edge of the flattened mesh 1204 and is in electrical communication with a power source (not shown) through a conductor 1210.

The flattened mesh 1204 is shaped to have an expanded configuration with an outside diameter 1212 greater than an outside diameter 1214 of the catheter 1202. The flattened mesh may comprise a material having shape memory. In such embodiments the flatted mesh may be formed with the expanded configuration being an unstressed state. A proximal end 1216 has a reduced diameter complementary to the outside diameter of the catheter 1202. The reduced diameter is secured to the catheter 1202. The flattened mesh 1204 tapers from the reduced diameter portion to the expanded diameter. As previously described, the conductive coating 1208 is applied to an edge of the flattened mesh 1204 which is twisted into a helix. A base material may be applied between the edge of the flattened mesh 1204 and the conductive coating 1208 to provide a surface for the conductive coating to adhere.

The flattened mesh 1208 may be changed from the expanded state of FIG. 12 to the radially constrained state of FIG. 13 by retracting the catheter 1202 into the sheath 1206 or advancing the sheath 1206 relative to the catheter 1202. The relative movement of the sheath 1206 and the catheter 1202 engages the taper of the flattened mesh 1204 providing an inward radial force to collapse the flattened mesh 1204. As the flattened mesh 1204 collapses, the sheath 1206 may further engage the taper of the flattened mesh 1204, constraining the flattened mesh 1204 and further collapsing it. Further movement of the sheath 1206 relative to the catheter 1202 may completely cover the flattened mesh 1204 collapsing it completely. In this collapsed state, the flattened mesh 1204 may be delivered to a treatment site.

The above figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

What is claimed:

1. A medical device comprising:
a first longitudinal member having a distal end and a proximal end;
a flattened mesh having a distal mesh end and a proximal mesh end, the flattened mesh being twisted to form a helix, the proximal mesh end being secured to the distal end of the first longitudinal member;
a conductor on an edge of the helix; and
a compression mechanism adapted to move the distal mesh end between a first position in which the flattened mesh is unexpanded and a second position in which the distal mesh end and the proximal mesh end are near one another thereby expanding the flattened mesh into an expanded state.

2. The medical device of claim 1 wherein the compression mechanism comprises a second longitudinal member disposed within the first longitudinal member, the second longitudinal member having a second longitudinal member distal end secured to the distal mesh end.

3. The medical device of claim 1 wherein the conductor comprises a conductive filament woven in the flattened mesh.

4. The medical device of claim 1 wherein the conductor comprises a conductive ink printed on the edge of the flattened mesh.

5. The medical device of claim 1 further comprising a second conductor on the edge of the helix, the second conductor being offset from the first conductive coating.

6. The medical device of claim 4 wherein the conductive ink covers the entire flattened mesh.

7. The medical device of claim 1 further comprising a radio frequency energy source in electrical communication with the conductor.

8. The medical device of claim 7 further comprising a radio frequency energy source in electrical communication with the conductor and the second conductor.

9. The medical device of claim 1 further comprising a second conductor disposed on an opposite edge of the helix.

10. The medical device of claim 9 wherein the helix extends for about half a turn.

11. A medical device comprising:
a catheter having a distal end and a first outer diameter at the distal end;
a flattened mesh having a distal mesh end and a proximal mesh end, the flattened mesh having an unstressed helical shape having a first helix outside diameter greater than the first outside diameter of the catheter, the proximal mesh end being secured to the distal end of the catheter;
a conductor disposed on an edge of the helical shape; and
a sleeve disposed about the distal end of the catheter, the sleeve having an inside surface having an inside diameter greater than the first outside diameter of the catheter and less than the helix outside diameter of the flattened mesh, the sleeve and catheter being slidable relative to each other from a first position in which the inside surface constrains the flattened mesh to have a second helix outer diameter less than the second outer diameter and a second position in which the inside surface does not constrain the flattened mesh.

12. The medical device of claim 11 wherein the sleeve extends to a proximal end of the catheter.

13. The medical device of claim 11 wherein the conductor comprises a conductive ink printed on the outer edge of the helix.

14. The medical device of claim 13 wherein the conductive ink coats the entire flattened mesh.

15. The medical device of claim 11 further comprising a radio frequency energy source in electrical communication with the conductor.

16. The medical device of claim 11 further comprising a second conductor disposed on the edge of the helix, the second conductor being offset from the first conductive coating.

17. The medical device of claim 16 further comprising a radio frequency energy source in electrical communication with the conductor and the second conductor.

18. The medical device of claim 11 wherein the flattened mesh is comprised of conductive filaments.

19. The medical device of claim 16 wherein the helix extends for about a half turn.

20. The medical device of claim 11 wherein the helix extends for about one full turn.
